(19) United States
(12) Patent Application Publication
(10) Pub. No.: US 2022/0362158 A1
(43) Pub. Date: Nov. 17, 2022

(54) COMPOSITION COMPRISING AT LEAST ONE TRITERPENE AND/OR AT LEAST ONE TRITERPENOID AND/OR AT LEAST ONE OF THE GLYCOSYLATED FORMS THEREOF

(71) Applicants: TILMAN SA, Baillonville (BE); ELEONOR SPRL, Waterloo (BE)

(72) Inventors: Yvan DIERCKXSENS, Overijse (BE); Céline MEINGUET, Ciney (BE); Jean-Noël TILMAN, Ernonheid (BE); Benoît WÉRY, Neupré (BE); Cristina LOIRA-PASTORIZA, Bruxelles (BE); Daniela MARTINEZ-TOVAR, Champigny-sur-Marne (BE); Fabian PRIEM, Waterloo (BE)

(21) Appl. No.: 17/622,128

(22) PCT Filed: Jun. 24, 2020
(86) PCT No.: PCT/EP2020/067742

§ 371 Date: Dec. 22, 2021

(30) Foreign Application Priority Data
Jun. 28, 2019 (BE) 2019/5419
Jul. 10, 2019 (BE) 2019/5448
Apr. 17, 2020 (BE) 2020/5254

(51) Int. Cl. A61K 9/20; A61K 36/324; A61K 36/68; A61K 31/704; A61K 31/19

(52) U.S. Cl. CPC A61K 9/2095; A61K 36/324; A61K 9/2013; A61K 9/2063; A61K 9/2059; A61K 36/68; A61K 31/704; A61K 31/19

(57) ABSTRACT

The present invention relates to a composition in the form of a thermoformed extrudate comprising at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase. The present invention also relates to the method for manufacturing by thermoforming such a composition and to the use thereof.

The present invention relates to a composition comprising at least one triterpene and/or at least one triterpenoid and/or at least one of the glycosylated forms thereof, to the manufacturing method thereof and to the use thereof.

Triterpenes and triterpenoids are widely distributed in the plant kingdom and have varied biological activities. The terms “triterpenes” and “triterpenoids” designate C30 (30 carbon atoms) substances of organic origin of the terpene family. Generally, the term “triterpene” is used to describe natural triterpenes and the broader term “triterpenoid” designates both natural triterpenes and the functionalized and/or metabolized derivatives thereof which maintain the triterpene structure. The classification into either of the categories of triterpenes and triterpenoids is not always clear in the literature and varies among authors. This is why the terms “triterpenes” and “triterpenoids” are frequently used to describe and designate the same C30 terpene compounds (Parmar and al., Neuropharmacological effects of triterpenoids. Phytopharmacology 2013, 4(2), 354-372). For example, some classifications consider boswellic acids to be pentacyclic triterpenoids while other classifications consider these same molecules to be pentacyclic triterpenes.

Two main categories are to be distinguished: tetracyclic triterpenes or triterpenoids and pentacyclic triterpenes or triterpenoids. Among the tetracyclic triterpenes or triterpenoids, there are in particular oleandrine, euphol and cucurbitacins. In the category of pentacyclic triterpenes or triterpenoids, there are in particular betulinic acid, oleanolic acid, boswellic acids, ursolic acid, lupeol, asiatic acid and maslinic acid.

Among the pentacyclic triterpenes or triterpenoids, the following groups of compounds are distinguished: hopane, arborane, fernan, gammaceran, onoceran, serratane, stictane, oleanane, ursane, taraxastane and lupane.

The tetracyclic triterpenes or triterpenoids and the pentacyclic triterpenes or triterpenoids constitute the most interesting groups of natural products because of their various pharmacological activities: anticancer, anti-inflammatory, analgesic, antimicrobial (antibacterial, antiviral, antifungal, antiplasmodic, . . . ), hepatoprotector and cardioprotector (antihypertensive, antidiabetic, antihyperlipidemic, antioxidant, . . . ), neuroprotector (antidepressant, antifatigue, actions against neurological disorders such as Parkinson's, Alzheimer's, . . . ).

In particular and without limitation, boswellic acids are powerful anti-inflammatories, inhibitors of the production of inflammation mediators, inhibitors of the activation of NF-kappaB, decrease interleukins IL-1, IL-2, IL-4, IL-6 and interferon-gamma; act on antibody production and cell-mediated immunity; inhibit 5-lipoxygenase (5-LOX) involved in the biosynthesis of leukotrienes by acting directly on an enzymatic site through the pentacyclic triterpene or triterpenoid structure and induce apoptosis of certain cancer cells by inhibiting topoisomerase. The anti-inflammatory properties of boswellic acids are in particular used for the treatment of several diseases where inflammation is involved, such as osteoarthritis, arthritis, rheumatoid arthritis, asthma, psoriasis or else chronic inflammatory intestinal diseases.

It should be noted that there are many α- and β-boswellic acids such as for example α-boswellic acid, acetyl-α-boswellic acid, β-boswellic acid, acetyl-β-boswellic acid, 9,11-dehydro-α-boswellic acid, acetyl-9,11-dehydro-α-boswellic acid, 9,11-dehydro-β-boswellic acid, acetyl-9,11-dehydro-β-boswellic acid, 11-keto-β-boswellic acid, 11-keto-α-boswellic acid, 3-acetyl-11-keto-α-boswellic acid or 3-acetyl-11-keto-β-boswellic acid.

It is widely recognized that triterpenes, triterpenoids and the glycosylated forms thereof, in particular that tetracyclic triterpenes or triterpenoids and that pentacyclic triterpenes or triterpenoids, are compounds which are very little or even completely insoluble in the water in which they do not disperse moreover only slightly or not at all, these compounds therefore having very low bioavailabilities. However, despite their low bioavailability/bioaccessibility (that is to say despite the small fraction of the administered dose which actually reaches the blood circulation in unchanged form), but also despite their low solubility and/or despite their low dispersion, in particular in the intestinal environment, the positive effects of triterpenes and triterpenoids and the glycosylated forms thereof, in particular of tetracyclic triterpenes or triterpenoids and of pentacyclic triterpenes or triterpenoids, on various pathologies make them molecules of interest for administration to the human being and/or for a veterinary use.

This is why numerous methodologies and numerous methods have been developed in order to formulate these compounds in the form of spherical particles, flakes, pellets or else granules. In particular, extrusion techniques can be used such as, for example, the extrusion-spheronization technique or the extrusion granulation technique (TSG or Tween Screw Granulation).

The extrusion-spheronization technique, as described for example in the document EP1391426, allows producing round particles of homogeneous size from a wet mass (containing an active substance and at least one excipient) which has passed through a grid having a predetermined mesh size before drying the particles thus obtained. More particularly, this powder shaping method is based on the following steps: mixture of an active substance and at least one excipient, wet granulation (compaction) of the previously obtained mixture, extrusion of the compacted mixture to obtain an extrudate, spheronization of the extrudate to form spherical particles/granules and drying of the obtained spherical particles/granules.

The extrusion granulation technique, as described for example in the document U.S. Pat. No. 5,260,074, for its part, allows obtaining intermediate products for the preparation of tablets and capsules. This technique is based on a granulation (compaction) of substances in the form of powders in an extruder to give rise to the formation of granules at the outlet of the latter.

Unfortunately, it appears that the current formulations comprising triterpenes and/or triterpenoids and/or the glycosylated forms thereof, in particular tetracyclic triterpenes or triterpenoids and/or pentacyclic triterpenes or triterpenoids, are inappropriate because of their too low or even of their non-solubility and/or because of their too low or even their non-dispersion in aqueous phase and/or because of the low release of these compounds from these formulations, which ultimately results in a low bioavailability/bioaccessibility of these molecules of interest. It should be noted that it also appears that the current methods for manufacturing these formulations are restrictive and difficult to implement.

The terms “dispersion in aqueous phase (in aqueous medium)”, refers, within the meaning of the present invention, to a system composed of two phases in which one of the two phases, called the dispersed phase, is finely divided in the other, called the dispersant phase. This dispersion can be molecular (solution), colloidal (dispersion of submicron particles) or coarser (dispersion of particles larger than one μm). More particularly, according to the invention, the term “dispersion in aqueous phase” designates suspensions consisting of a solid phase dispersed in an aqueous phase (liquid).

Within the meaning of the present invention, the term “solubility” designates the ability of a substance, called a solute, to dissolve in another substance, called a solvent, to form a homogeneous mixture called a solution.

The object of the invention is to overcome at least partially the drawbacks of the state of the art by providing (1) a composition comprising at least one triterpene and/or at least one triterpenoid and/or at least one of the glycosylated forms thereof whose solubility (solubilities) and/or dispersion(s) in aqueous phase (aqueous medium) is (are) increased such that the bioavailability (bioavailabilities) of these compounds is (are) significantly increased and (2) a method for manufacturing such a composition which is easy to implement, which is flexible, which is economically profitable and which ensures that said at least one triterpene and/or said at least one triterpenoid and/or said at least one of the glycosylated forms thereof is(are) present and homogeneously distributed in the obtained final composition.

Moreover, the invention intends to provide a composition which is stable over time, that is to say which retains its properties in terms of solubility and/or dispersion of said at least one triterpene and/or of said at least one triterpenoid and/or of said at least one of the glycosylated forms thereof and which retains its properties in terms of the rate of release of these compounds over time from a composition (formulation) according to the invention, this in particular in aqueous phase and more particularly in the intestinal environment.

In order to at least partially resolve these problems, a composition is provided, according to the invention, in the form of a thermoformed extrudate comprising at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.

In particular, according to the invention, said at least one polymer is a thermoplastic polymer, that is to say a polymer having the property of being softened when it is heated sufficiently, but which, upon cooling, becomes hard again.

According to one embodiment, there is therefore provided, according to the invention, a composition in the form of a thermoformed extrudate comprising at least one triterpene and/or at least one triterpenoid and/or at least one of the glycosylated forms thereof and at least one natural or synthetic protein as polymer, said at least one triterpene and/or said at least one triterpenoid and/or said at least one of the glycosylated forms thereof comprising at least one first amorphous phase and optionally one second crystalline phase.

According to one embodiment, there is also provided, according to the invention, a composition in the form of a thermoformed extrudate comprising at least one triterpene and/or at least one triterpenoid and/or at least one of the glycosylated forms thereof and at least one natural or synthetic oligosaccharide as polymer, said at least one triterpene and/or said at least one triterpenoid and/or said at least one of the glycosylated forms thereof comprising at least one first amorphous phase and optionally one second crystalline phase.

According to one embodiment, there is also provided, according to the invention, a composition in the form of a thermoformed extrudate comprising at least one triterpene and/or at least one triterpenoid and/or at least one of the glycosylated forms thereof and at least one natural or synthetic polysaccharide as polymer, said at least one triterpene and/or said at least one triterpenoid and/or said at least one of the glycosylated forms thereof comprising at least one first amorphous phase and optionally one second crystalline phase.

The term “extrudate”, refers, within the meaning of the present invention, to a material which comes out of an extruder, in particular of the die of an extruder.

The term “thermoformed extrudate”, refers, within the meaning of the present invention, to a material which comes out of an apparatus, in particular which comes out of an extruder, in which it has undergone a transformation by the effect of heat, optionally by the combined effect of heat and shear forces of a worm screw. Such a transformation by the effect of heat, optionally by the combined effect of heat and shear forces of a worm screw, can be obtained with the technique of hot extrusion (HME or Hot Melt Extrusion).

In particular, a thermoformed extrudate according to the invention is an extrudate in which the active ingredient(s) (said at least one triterpene and/or said at least one triterpenoid and/or said at least one of the glycosylated forms thereof) and/or said at least one polymer is/are melted. In particular, the composition according to the invention, more particularly the composition in the form of a thermoformed extrudate according to the invention, is a solid dispersion in which said at least one triterpene and/or said at least one triterpenoid and/or said at least one of the glycosylated forms thereof comprising at least one first amorphous phase and optionally one second crystalline phase is/are dispersed in said at least one polymer selected from the group consisting of natural or synthetic proteins, natural or synthetic oligosaccharides, natural or synthetic polysaccharides, the derivatives thereof and the mixtures thereof.

According to one embodiment, there is therefore provided, according to the invention, a composition in the form of a thermoformed extrudate comprising at least one triterpene and/or at least one triterpenoid and/or at least one of the glycosylated forms thereof and at least one natural or synthetic protein as polymer, said at least one triterpene and/or said at least one triterpenoid and/or said at least one of the glycosylated forms thereof comprising at least one first amorphous phase and optionally one second crystalline phase, said composition being a solid dispersion in which said at least one triterpene and/or said at least one triterpenoid and/or said at least one of the glycosylated forms thereof is/are dispersed in said at least one natural or synthetic protein as polymer, which is/has been melted.

According to one embodiment, there is also provided, according to the invention, a composition in the form of a thermoformed extrudate comprising at least one triterpene and/or at least one triterpenoid and/or at least one of the glycosylated forms thereof and at least one natural or synthetic oligosaccharide as polymer, said at least one triterpene and/or said at least one triterpenoid and/or said at least one of the glycosylated forms thereof comprising at least one first amorphous phase and optionally one second crystalline phase, said composition being a solid dispersion in which said at least one triterpene and/or said at least one triterpenoid and/or said at least one of the glycosylated forms thereof is/are dispersed in said at least one natural or synthetic oligosaccharide as polymer, which is/has been melted.

According to one embodiment, there is also provided, according to the invention, a composition in the form of a thermoformed extrudate comprising at least one triterpene and/or at least one triterpenoid and/or at least one of the glycosylated forms thereof and at least one natural or synthetic polysaccharide as polymer, said at least one triterpene and/or said at least one triterpenoid and/or said at least one of the glycosylated forms thereof comprising at least one first amorphous phase and optionally one second crystalline phase, said composition being a solid dispersion in which said at least one triterpene and/or said at least one triterpenoid and/or said at least one of the glycosylated forms thereof is/are dispersed in said at least one natural or synthetic polysaccharide as polymer, which is/has been melted.

A thermoformed extrudate according to the invention is obtained by hot thermoforming, in particular by hot thermoforming using the hot extrusion technique. According to the invention, the hot thermoforming therefore relates more particularly to the hot extrusion technique.

There is therefore provided, according to the invention, a composition obtained by hot thermoforming in the form of a thermoformed extrudate obtained by hot thermoforming, in particular obtained by hot extrusion, said composition comprising at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.

A thermoformed extrudate according to the invention can therefore be obtained according to the hot extrusion technique which allows achieving the molecular dispersion of an active agent (active substance) within a polymer matrix (within a polymer) to form solid dispersions. This solid dispersion is possible thanks to a heat supply and possibly thanks to the stress applied by the movement of the worm screws on the material in an extruder. Finally, the hot extrusion results, at the outlet, in the formation of a thermoformed extrudate in the form of a strand which can then in particular be pelletized or crushed.

If the extrusion-spheronization technique and the extrusion granulation technique as described above are carried out without heat supply (heating) and they typically require a liquid phase (generally water) to obtain spherical particles and/or granules, the hot extrusion technique can be carried out without supplying this liquid phase but relies on heat supply (heating) to ensure a transformation of the material by thermoforming.

Moreover, if the extrusion-spheronization technique and the extrusion granulation technique consist of an agglomeration into powder granules while trying as much as possible to retain the initial properties of the constituents of these powders, the hot extrusion technique results in, instead, a transformation of the material, in particular a glassy structure obtained under the action of heat (heating), the particles constituting the powders being no longer all present in their crystalline initial (native) form at the end of the hot extrusion process but having undergone a transformation by thermoforming.

The terms “comprising at least one first amorphous phase and optionally one second crystalline phase”, mean, within the meaning of the present invention, that said at least one triterpene and/or said at least one triterpenoid and/or said at least one of the glycosylated forms thereof can either comprise 100% by mass of an amorphous phase, or can simultaneously comprise one first amorphous phase and one second crystalline phase, the sum of the percentages by mass of the first and second phases being in this case equal to 100. In other words, the composition according to the invention can comprise said at least one triterpene and/or said at least one triterpenoid and/or said at least one of the glycosylated forms thereof (1) totally in amorphous form or (2) partly in amorphous form (phase) and partly in crystalline form (phase).

It should be noted that a phase is called amorphous when the atoms constituting this phase do not respect any order at medium and long distance, which distinguishes it from a so-called crystalline phase.

The composition according to the invention is therefore in the form of a thermoformed extrudate in which said at least one triterpene and/or said at least one triterpenoid and/or said at least one of the glycosylated forms thereof (active ingredient) comprises (comprise) at least one first amorphous phase and optionally one second crystalline phase, which phase(s) is/are dispersed within at least one polymer selected from the group consisting of natural or synthetic proteins, natural or synthetic oligosaccharides, natural or synthetic polysaccharides, the derivatives thereof and the mixtures thereof.

The term “natural protein”, refers, within the meaning of the present invention, to any protein naturally present in the living world, in particular a plant or animal protein.

The term “synthetic protein”, refer, within the meaning of the present invention, to any protein subject to human intervention, in particular to a protein obtained at the start of a chemical or biochemical or biotechnological process.

The terms “natural oligosaccharides” and “natural polysaccharides”, refer, within the meaning of the present invention, to any oligosaccharide and any polysaccharide naturally present in the living world, in particular to an oligosaccharide or to a plant or animal polysaccharide.

The terms “synthetic oligosaccharides” and “synthetic polysaccharides”, refer, within the meaning of the present invention, to any oligosaccharide and any polysaccharide which is the subject to human intervention, in particular to any oligosaccharide and to any polysaccharide obtained at the start of a chemical or biochemical or biotechnological process.

It has been determined, in the context of the present invention, that such a composition in the form of a thermoformed extrudate comprising at least one triterpene and/or at least one triterpenoid and/or at least one of the glycosylated forms thereof as active ingredient(s) and at least one polymer selected from the group consisting of natural or synthetic proteins, natural or synthetic oligosaccharides, natural or synthetic polysaccharides, the derivatives thereof and the mixtures thereof and in which said at least one triterpene and/or said at least one triterpenoid and/or said at least one of the glycosylated forms thereof comprises (comprise) at least one first amorphous phase and optionally one second crystalline phase has distinctly higher solubility (solubilities) and/or dispersion(s) in aqueous phase (aqueous medium) of said at least one triterpene and/or of said at least one triterpenoid and/or of said at least one of the glycosylated forms thereof.

Moreover, it has been shown that such a composition according to the invention has significantly increased bioavailability (bioavailabilities)/bioaccessibility (bioaccessibilities) of said at least one triterpene and/or of said at least one triterpenoid and/or or of said at least one of the glycosylated forms thereof with respect to the bioavailabilities/bioaccessibilities observed for the current compositions.

According to the invention, the active ingredient(s), that is to say said at least one triterpene and/or said at least one triterpenoid and/or said at least one of the glycosylated forms thereof comprising at least one first amorphous phase and optionally one second crystalline phase, is (are) homogeneously dispersed/spread/distributed within at least one polymer selected from the group consisting of natural or synthetic proteins, natural or synthetic oligosaccharides, natural or synthetic polysaccharides, the derivatives thereof and the mixtures thereof, the active ingredient(s) and/or said at least one polymer being melted during the thermoforming manufacturing method implemented according to the invention and described later.

In addition, a composition according to the invention can be stored for several months without its properties being altered. In particular, it has been demonstrated that a composition according to the invention retains its properties in terms of solubility (solubilities) and/or dispersion(s) in aqueous phase (aqueous medium) of said at least one triterpene and/or of said at least one triterpenoid and/or of said at least one of the glycosylated forms thereof and in terms of the rate of release of this (these) compound(s) over time from a composition/formulation according to the invention, this in particular in the aqueous phase.

Advantageously, according to the invention, said thermoformed extrudate comprises a thermoformed mixture of said at least one triterpene and/or of said at least one triterpenoid and/or of said at least one of the glycosylated forms thereof and of said at least one polymer selected from the group consisting of natural or synthetic proteins, natural or synthetic oligosaccharides, natural or synthetic polysaccharides, the derivatives thereof and the mixtures thereof.

Alternatively, according to the invention, said thermoformed extrudate consists of a thermoformed mixture of at least one triterpene and/or of at least one triterpenoid and/or of at least one of the glycosylated forms thereof and of at least one polymer selected from the group consisting of natural or synthetic proteins, natural or synthetic oligosaccharides, natural or synthetic polysaccharides, the derivatives thereof and the mixtures thereof.

There is therefore provided, according to the invention, a thermoformed extrudate obtained by hot thermoforming, in particular obtained by hot extrusion, said thermoformed extrudate comprising a thermoformed mixture of at least one triterpene and/or of at least one triterpenoid and/or of at least one of the glycosylated forms thereof and of at least one polymer selected from the group consisting of natural or synthetic proteins, natural or synthetic oligosaccharides, natural or synthetic polysaccharides, the derivatives thereof and the mixtures thereof.

The term “thermoformed mixture”, refers, within the meaning of the present invention, to a mixture which comes out of an apparatus, in particular which comes out of an extruder, in which it has undergone a transformation by the effect of heat, optionally by the combined effect of heat and of shear forces of a worm screw. Such a transformation by the effect of heat, optionally by the combined effect of heat and shear forces of a worm screw, can be obtained with the hot extrusion technique (HME).

In particular, a thermoformed mixture according to the invention is a mixture in which the active ingredient(s) (said at least one triterpene and/or said at least one triterpenoid and/or said at least one of the glycosylated forms thereof) and/or said at least one polymer is/are melted/was/were melted.

According to the invention, said at least one triterpene and/or said at least one triterpenoid and/or said at least one of the glycosylated forms thereof comprises (comprise) one first amorphous phase.

Advantageously, according to the invention, said at least one triterpene and/or said at least one triterpenoid and/or said at least one of the glycosylated forms thereof comprises (comprise) predominantly at least one first amorphous phase.

Preferably, according to the invention, said at least one triterpene and/or said at least one triterpenoid and/or said at least one of the glycosylated forms thereof comprises between 51 and 100% by mass of an amorphous phase and between 0 and 49% by mass of a crystalline phase.

The terms “predominantly at least one first amorphous phase”, means, within the meaning of the present invention, that said at least one triterpene and/or said at least one triterpenoid and/or said at least one of the glycosylated forms thereof comprises (comprise) between 50 and 100% by mass of an amorphous phase and between 0 and 50% of a crystalline phase, more particularly that said at least one triterpene and/or said at least one triterpenoid and/or said at least one of the glycosylated forms thereof comprises (comprise) between 51 and 100% by mass of an amorphous phase and between 0 and 49% of a crystalline phase.

Advantageously, according to the invention, said at least one triterpene and/or said at least one triterpenoid and/or said at least one of the glycosylated forms thereof is tetracyclic, such as for example oleandrine, euphol or cucurbitacin, or pentacyclic, such as, for example, betulinic acid, oleanolic acid, boswellic acid, ursolic acid, lupeol, asiatic acid, madecassic acid, maslinic acid, jujubogenin or pseudojujubogenin.

The glycosylated forms of triterpenes, such as, for example, ginsenosides, also fall within the scope of the present invention. The glycosylated forms of triterpenoids, such as, for example, bacosides, asiaticosides and α-hederin also fall within the scope of the present invention.

Advantageously, according to the invention, said boswellic acid is selected from the group consisting of α- and β-boswellic acids, for example α-boswellic acid, acetyl-α-boswellic acid, β-boswellic acid, acetyl-β-boswellic acid, 9,11-dehydro-α-boswellic acid, acetyl-9,11-dehydro-α-boswellic acid, 9,11-dehydro-β-boswellic acid, acetyl-9,11-dehydro-β-boswellic acid, 11-keto-β-boswellic acid, 11-keto-α-boswellic acid, 3-acetyl-11-keto-α-boswellic acid, 3-acetyl-11-keto-β-boswellic acid.

Preferentially, according to the invention, said natural or synthetic proteins are selected from the group consisting of glycoproteins, collagens and/or collagen hydrolysates, plant proteins, animal proteins, the derivatives thereof and the mixtures thereof.

The term “collagen hydrolyzate”, refers, within the meaning of the present invention, to gelatins and hydrolyzed collagens or collagen peptides. The terms “collagen hydrolyzate” therefore encompass gelatins having the ability to gel, but also hydrolyzed collagens or collagen peptides which have or not the ability to gel.

By way of example, when said at least one natural or synthetic protein is collagen or gelatin, it may be collagen or gelatin of animal origin (fish, pork, beef, . . . ).

By way of example, when said at least one natural or synthetic protein is a plant protein, it may be a soybean, pumpkin, rice, wheat, pea or nut protein. This list is not exhaustive.

Preferentially, according to the invention, said collagens and/or said collagen hydrolysates have a molecular weight comprised between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

Advantageously, according to the invention, said collagens and/or said collagen hydrolysates have a molecular weight comprised between 1000 and 300000 Da, preferably between 1500 and 150000 Da, preferably between 2000 and 60000 Da.

Preferably, according to the invention, said collagens and/or said collagen hydrolysates have a molecular weight equal to 50 Da or equal to 100 Da or equal to 150 Da or equal to 200 Da or equal to 250 Da or equal to 300 Da or equal to 350 Da or equal to 400 Da or equal to 450 Da or equal to 500 Da or equal to 550 Da or equal to 600 Da or equal to 650 Da or equal to 700 Da or equal to 750 Da or equal to 800 Da or equal to 850 Da or equal to 900 or equal to 950 Da or equal to 1000 Da or equal to 1100 Da or equal to 1200 or equal to 1300 Da or equal to 1400 Da or equal to 1500 Da or equal to 1600 Da or equal to 1700 Da or equal to 1800 Da or equal to 1900 Da or equal to 2000 Da or equal to 2500 Da or equal to 3000 Da or equal to 3500 Da or equal to 4000 Da or equal to 4500 Da or equal to 5000 Da or equal to 5500 Da or equal to 6000 Da or equal to 6500 Da or equal to 7000 Da or equal to 7500 Da or equal to 8000 Da or equal to 8500 Da or equal to 9000 Da or equal to 9500 Da or equal to 10000 Da or equal to 12500 Da or equal to 15000 Da or equal to 17500 Da or equal to 20000 Da or equal to 22500 Da or equal to 25000 Da or equal to 27500 Da or equal to 30000 Da or equal to 32500 Da or equal to 35000 Da or equal to 37500 Da or equal to 40000 Da or equal to 42500 Da or equal to 45000 Da or equal to 47500 Da or equal to 50000 Da or equal to 55000 Da or equal to 60000 Da or equal to 65000 Da or equal to 70000 Da or equal to 75000 Da or equal to 80000 Da or equal to 85000 Da or equal to 90000 Da or equal to 100000 Da or equal to 110000 Da or equal to 120000 Da or equal to 130000 Da or equal to 140000 Da or equal to 150000 Da or equal to 160000 Da or equal to 170000 Da or equal to 180000 Da or equal to 190000 Da or equal to 200000 Da or equal to 210000 Da or equal to 220000 Da or equal to 230000 Da or equal to 240000 Da or equal to 250000 Da or equal to 260000 Da or equal to 270000 Da or equal to 280000 Da or equal to 290000 Da or equal to 300000 Da.

According to one embodiment according to the invention, when said at least one natural or synthetic protein is collagen, the latter has a molecular weight comprised between 900 and 7000 Da, preferably a molecular weight comprised between 950 and 5000 Da, preferentially a molecular weight comprised between 1000 and 3000 Da.

According to one embodiment according to the invention, when said at least one natural or synthetic protein is collagen, the latter has a molecular weight equal to 2000 Da or equal to 3000 Da or equal to 5000 Da or equal to 50000 Da.

According to one embodiment according to the invention, when said at least one natural or synthetic protein is gelatin, the latter has a molecular weight comprised between 900 and 6000 Da, preferably a molecular weight comprised between 950 and 5000 Da, preferentially a molecular weight comprised between 1000 and 3000 Da.

According to one embodiment according to the invention, when said at least one natural or synthetic protein is gelatin, the latter has a molecular weight equal to 2000 Da or equal to 3000 Da or equal to 5000 Da or equal to 50000 Da.

According to one embodiment according to the invention, when said at least one natural or synthetic protein is a hydrolyzed collagen or a collagen peptide, the latter has a molecular weight comprised between 900 and 6000 Da, preferably a molecular weight comprised between 950 and 5000 Da, preferentially a molecular weight comprised between 1000 and 3000 Da.

According to one embodiment according to the invention, when said at least one natural or synthetic protein is a hydrolyzed collagen or a collagen peptide, the latter has a molecular weight equal to 2000 Da or equal to 3000 Da or equal to 5000 Da or equal to 50000 Da.

Preferably, according to the invention, said oligosaccharides are selected from the group consisting of cyclodextrin, raffinose, rhamminose, rhamnose, stachyose, verbascose, trehalose, lactose, lactulose, maltose, the derivatives thereof and the mixtures thereof.

Advantageously, according to the invention, said natural or synthetic polysaccharides are selected from the group consisting of starches (corn starch, potato starch, pregelatinized starch, . . . ), fibers (acacia, inulin, alginates, carrageenans, pectin, . . . ), celluloses and hemicelluloses (for example hydroxypropylcellulose (HPC), hydroxypropylmethylcellulose phthalate, hydroxypropymethylcellulose (HPMC), hydroxypropymethylcellulose acetate succinate, cellulose acetate butyrate, cellulose acetate phthalate), glycogen, β-glucan, inulin, amylopectin, amylose, dextrin, maltodextrin, isomaltose, xylan, pullulan, agar-agar, carrageenans, mannans, fucoidan, gums (xanthan, guar, mastic or gum arabic, . . . ), chitosan, chitin, xanthan, levan, neoserine, hyaluronic acid, hyaluronates, chondroitin sulfate, dermatan sulfate, keratan sulfate, the derivatives thereof and the mixtures thereof.

According to one embodiment, the composition according to the invention further comprises at least one additional natural or synthetic polymer selected from the group consisting of polyvinyl acetate, polyvinylpyrrolidone (PVP), polyvinylpyrrolidone-co-vinyl acetate, polyethylene-co-vinyl acetate, polyvinyl acid co-methacrylic acetate, polyethylene oxide, polylactide-co-glycolide, polyvinyl alcohol, polycarbophil, polycaprolactone, carnauba wax, ethylene-vinyl copolymer, lecithin, castor oil, hydrogenated soybean oil, waxes, isomalt, the derivatives thereof and the mixtures thereof.

Advantageously, the composition according to the invention further comprises at least one plasticizer. The addition of a plasticizer in a composition according to the invention allows obtaining a composition according to the invention through a manufacturing method where temperatures below the melting points of said at least one triterpene and/or of said at least one triterpenoid and/or of said at least one of the glycosylated forms thereof and of the polymer can be used in order to guarantee all the same a melting of these two compounds and the dispersion/spread/distribution of said at least one triterpene and/or of said at least one triterpenoid and/or of said at least one of the glycosylated forms thereof within the polymer

Preferentially, according to the invention, said plasticizer is selected from the group consisting of polyols (glycerol, sorbitol, mannitol, . . . ), lipids (fatty acids, fatty acid esters, fatty acid amides, glycerides, phospholipids, . . . ), sucrose esters, water, triethyl citrate, polyethylene glycol, dibutyl sebate, butyl stearate, glycerol monostearate, diethyl phthalate, the derivatives thereof and the mixtures thereof.

For example, according to the invention, the phospholipids can be lecithins such as soya lecithin, sunflower lecithin or egg yolk lecithin.

According to the invention, the preferred plasticizers are glycerol, water, polyethylene glycol and triethyl citrate.

Preferably, the composition according to the invention further comprises at least one additive selected from the group consisting of lubricants, surfactants, antioxidants, chelants, the derivatives thereof and the mixtures thereof.

By way of example, the following compounds can be used, alone or as a mixture, as lubricants in a composition according to the invention: glycerol dibhenate, talc, silica, stearic acid, boric acid, waxes, sodium oleate, sodium acetate, magnesium stearate, calcium stearate, sodium stearate, sodium benzoate, sodium lauryl sulfate, glycerol distearate, glycerol palmitostearate, microcrystalline cellulose or even polyoxyl-8-glycerides.

By way of example, the following compounds can be used, alone or as a mixture, as surfactants in a composition according to the invention: Pluronic®, Span®, Cremophor®, polysorbates (Tween®, . . . ), vitamin E TPGS and sodium ducosate.

By way of example, the following compounds can be used, alone or as a mixture, as antioxidants and/or chelants in a composition according to the invention: butylated hydroxytoluene, butylated hydroxyanisiole, EDTA, citric acid and vitamin E.

Advantageously, the composition according to the invention further comprises at least one additional compound of polyphenol type selected from the group consisting of phenolic acids, stilbenes, phenolic alcohols, lignans, flavonoids, the derivatives thereof and the mixtures thereof. In particular, the glycosylated and aglycone forms of the polyphenols are envisaged as an additional active ingredient according to the present invention. More particularly, within the meaning of the present invention, the term “polyphenol” designates both polyphenols of natural origin and synthetic polyphenols but also all polyphenol derivatives.

By way of example, within the meaning of the present invention, derivatives of hydroxybenzoic acid (gallic acid, tannic acid, . . . ) and derivatives of hydroxycinamic acid (curcumin, coumaric acid, caffeic acid, ferulic acid, . . . ) may be mentioned as phenolic acids.

By way of example, within the meaning of the present invention, resveratrol, sirtinol, piceatannol or else polydatin may be mentioned as stilbenes.

By way of example, within the meaning of the present invention, flavanoles (quercetin, myricetin, kaempferol, isorhamnetin, morine, rutin, tiliroside, trihydroxyethylrutin, fisetin, . . . ), flavones (apigenin, luteolin, baicalein, chrysin, diosmin, nobiletin, tangeretin, wogonin, aminogenistein, . . . ), flavanones (bavachine, 8-isopentenylnaringenin, isoxanthohumole, naringenin, eriodictyole, hesperetin, silybin, taxifolin, . . . ), isoflavones (genistein, daidzein, daidzin, formonetin, genistin, neobavaoflavone, pueranin, . . . ), antocianidins (cianidin, pelargonidin, delphinidin, petunidin, malvidin, . . . ) and flavanols (cathechins, gallocatechin, epigallocatechingallate, . . . ) may be mentioned as flavonoids.

According to the invention, said at least one additional active ingredient of polyphenol type constitutes an inhibitor/modulator of efflux pumps including P-gp.

Preferentially, the composition according to the invention further comprises at least one inhibitor and/or a modulator of the activity of P-gp.

Preferably, the composition according to the invention is packaged in the form of pellets, flakes, granules, powders, effervescent or non-effervescent tablets, injectable or non-injectable solutions, suspensions, gels, ointments or even in any other form suitable for administration to an animal or to a human being.

Other embodiments of a composition according to the invention are indicated in the appended claims.

An object of the invention is also a manufacturing method, in particular a method for manufacturing by thermoforming a composition in the form of a thermoformed extrudate according to the invention, characterized in that it comprises the following steps:
a) a step of simultaneous or delayed supplying at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, to be fed into an extruder,
b) a step of mixing, in said extruder, said at least one triterpene and/or said at least one triterpenoid and/or said at least one of the glycosylated forms thereof and said at least one polymer selected from the group consisting of natural or synthetic proteins, natural or synthetic oligosaccharides, natural or synthetic polysaccharides, the derivatives thereof and the mixtures thereof, to form a mixture, and
c) a step of hot extruding said mixture obtained in step b) in said extruder to obtain a thermoformed extrudate in which said at least one triterpene and/or said at least one triterpenoid and/or said at least one of the glycosylated forms thereof comprises/comprise at least one first amorphous phase and optionally one second crystalline phase.

Such a method according to the invention gives rise to a composition in the form of a thermoformed extrudate, that is to say obtained by thermoforming and more particularly by hot extrusion, in which said at least one triterpene and/or said au at least one triterpenoid and/or said at least one of the glycosylated forms thereof as active ingredient(s) comprises (comprise) at least one first amorphous phase and optionally one second crystalline phase dispersed within said at least one polymer selected from the group consisting of natural or synthetic proteins, natural or synthetic oligosaccharides, natural or synthetic polysaccharides, the derivatives thereof and the mixtures thereof. This composition according to the invention has a significantly higher solubility (solubilities) and/or dispersion(s) in aqueous phase (aqueous medium) of said at least one triterpene and/or of said at least one triterpenoid and/or of said at least one of the glycosylated forms thereof and simultaneously a significantly increased bioavailability (bioavailabilities) of said at least one triterpene and/or of said at least one triterpenoid and/or of said at least one of the glycosylated forms thereof relative to the solubilities and bioavailabilities of these compounds for the current compositions. It has been shown that the composition according to the invention is in the form of a thermoformed extrudate in which said at least one triterpene and/or said at least one triterpenoid and/or said at least one of the glycosylated forms thereof (active ingredient(s)) comprising at least one first amorphous phase and optionally one second crystalline phase is (are) dispersed within said at least one polymer selected from the group consisting of natural or synthetic proteins, natural or synthetic oligosaccharides, natural or synthetic polysaccharides, the derivatives thereof and the mixtures thereof.

It has also been shown, in the context of the present invention, that said at least one triterpene and/or said at least one triterpenoid and/or said at least one of the glycosylated forms thereof is (are) not degraded even during the manufacturing method, in particular during the method for manufacturing by thermoforming the composition in the form of a thermoformed extrudate which nevertheless involves the submission of said at least one triterpene and/or of said at least one triterpenoid and/or of said at least one of the glycosylated forms thereof to high temperatures (HME). Moreover, it has also been demonstrated that said at least one triterpene and/or said at least one triterpenoid and/or said at least one of the glycosylated forms thereof in a composition in the form of a thermoformed extrudate according to the invention is (are) homogeneously spread (distributed) therein.

More particularly, the hot extrusion (Hot Melt Extrusion—HME) carried out according to the thermoforming manufacturing method according to the invention gives rise to a melting of the active ingredient(s) (said at least one triterpene and/or said at least one triterpenoid and/or said at least one of the glycosylated forms thereof) and/or of said at least one polymer at a temperature greater than or equal to their melting point.

However, according to certain embodiments of a composition according to the invention, this melting of the active ingredient(s) and/or of the polymer can take place at a temperature below their melting point. This is the case, for example, if the composition according to the invention comprises a plasticizer or if the active ingredient(s) itself (themselves) has (have) plasticizing properties. Such a melting of the active ingredient(s) and/or of the polymer gives rise to a solid dispersion in which the active ingredient(s) (said at least one triterpene and/or said at least one triterpenoid and/or said at least one of the glycosylated forms thereof) comprising at least one first amorphous phase and optionally one second crystalline phase is (are) homogeneously dispersed/spread/distributed within said at least one polymer selected from the group consisting of natural or synthetic proteins, natural or synthetic oligosaccharides, natural or synthetic polysaccharides, the derivatives thereof and the mixtures thereof.

Advantageously, the method according to the invention comprises a preliminary step of premixing said at least one triterpene and/or said at least one triterpenoid and/or said at least one of the glycosylated forms thereof and said at least one polymer selected from the group consisting of natural or synthetic proteins, natural or synthetic oligosaccharides, natural or synthetic polysaccharides, the derivatives thereof and the mixtures thereof, in such a way to form a premixture intended to be fed into the extruder.

Preferably, according to the method according to the invention, said hot extrusion step is carried out at an extrusion temperature or a thermoforming temperature comprised between 20 and 300° C., preferably at a temperature between 40 and 270° C., preferentially at a temperature comprised between 50 and 250° C., preferably at a temperature comprised between 60 and 230° C., more preferentially at a temperature comprised between 70 and 220° C., more preferentially at a temperature comprised between 80 and 200° C., more preferentially at a temperature comprised between 90 and 180° C., more preferentially at a temperature comprised between 100 and 170° C., more preferentially at a temperature comprised between 120 and 160° C., more preferentially at a temperature comprised between 125 and 150° C.

Advantageously, according to the method according to the invention, said hot extrusion step is carried out at a rotation speed of an extrusion screw comprised between 20 and 900 rpm, preferably comprised between 50 and 300 rpm, preferably comprised between 100 and 250 rpm, preferentially equal to 250 rpm, preferentially equal to 100 rpm.

Preferentially, the method according to the invention comprises an additional step of cooling at the outlet of the extruder.

Advantageously, the method according to the invention comprises an additional step of treating the thermoformed extrudate at the outlet of the extruder, for example cutting at a pelletizer and/or grinding said thermoformed extrudate.

Other embodiments of the method according to the invention are indicated in the appended claims.

The present invention also relates to a composition in the form of a thermoformed extrudate obtained according to the method according to the invention, said composition comprising at least one triterpene and/or at least one triterpenoid and/or at least one of the glycosylated forms thereof as active ingredient and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.

In other words, the present invention also relates to a composition in the form of a thermoformed extrudate obtained by thermoforming, in particular by hot extrusion (HME—Hot Melt Extrusion), said composition comprising at least one triterpene and/or at least one triterpenoid and/or at least one of the glycosylated forms thereof as active ingredient and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.

The present invention also relates to a use of a composition according to the invention as a food supplement and/or as a cosmetic and/or as a medicament for human or veterinary use.

A composition according to the invention preferentially has anti-inflammatory, hypolipidemic, antioxidant, antithrombotic, antitumor and antidiabetic properties as well as neuroprotective properties.

In particular, the present invention relates to a composition for use in the preventive and/or curative treatment, in human and/or in animal, of pathologies related to inflammation (osteoarthritis, tendonitis, injuries, . . . ), pathologies related to the premature aging of cells, pathologies related to the cardiovascular system (hypotension, hypertension, vasoconstriction, ventricular hypertrophy, arrhythmia, hepatic steatosis, . . . ), pathologies related to the blood system (cholesterolemia, platelet aggregation, . . . ), pathologies related to the gastrointestinal system (diarrhea, digestive inflammation, modulation of the intestinal microbiota, . . . ), pathologies related to the endocrine system (hyperglycemia, . . . ), pathologies related to the immune system, pathologies related to the central nervous system, skin diseases, diseases due to the presence of microorganisms and cancers (anti-tumor, . . . ) and in the preventive and/or curative treatment of diabetes.

More particularly, the present invention relates to a composition for use in the preventive and/or curative treatment, in human and/or in animal, of diseases associated with the joints, muscles and tendons, diseases associated with premature aging of cells, obesity, diabetes, hypercholesterolemia, metabolic syndrome and irritable bowel syndrome (IBS).

Other forms of use of a composition according to the invention are indicated in the appended claims.

Other characteristics, details and advantages of the invention will emerge from the examples given below, not by way of limitation and with reference to the appended figures.

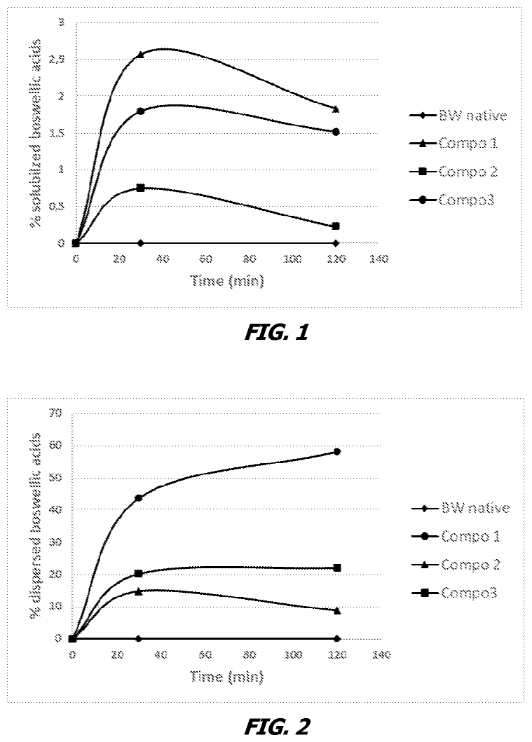

FIG. 1 is a graph illustrating the rate of solubilization of boswellic acids over time for different examples of compositions, in particular for different examples of thermoformed compositions, according to the invention.

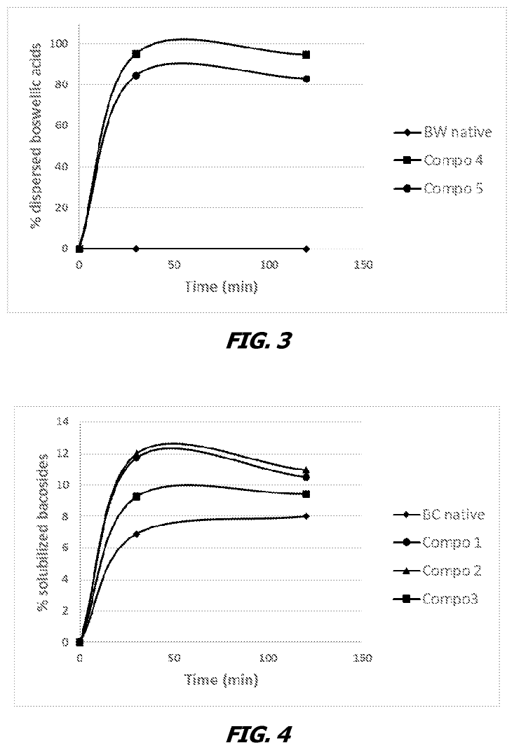

FIGS. 2 and 3 are graphs illustrating the rates of dispersion over time of examples of different compositions, in particular for examples of different thermoformed compositions, according to the invention.

FIG. 4 is a graph illustrating the rate of solubilization of bacosides over time for different examples of compositions, in particular for different examples of thermoformed compositions, according to the invention.

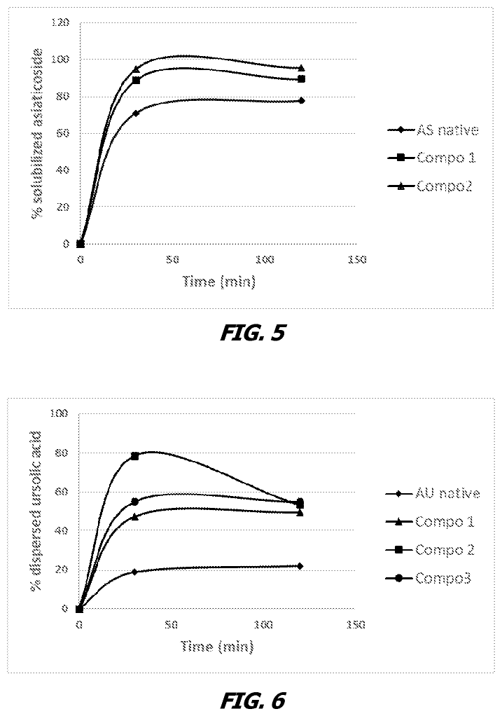

FIG. 5 is a graph illustrating the rate of solubilization of asiaticoside over time for different examples of compositions, in particular for different examples of thermoformed compositions, according to the invention.

FIG. 6 is a graph illustrating the rate of dispersion of ursolic acid over time for different examples of compositions, in particular for different examples of thermoformed compositions, according to the invention.

### EXAMPLES

### Example 1: Thermoforming Manufacturing Method of a Composition According to the Invention in the Form of a Thermoformed Extrudate

Thermoformed compositions according to the invention comprising at least one triterpene and/or at least one triterpenoid and/or at least one of the glycosylated forms thereof comprising at least one amorphous phase, such as those which are the subject of Example 2 below, were obtained according to the following method which is also the subject of the present invention:
a) a step of premixing at least one triterpene and/or at least one triterpenoid and/or at least one of the glycosylated forms thereof in the crystalline state in powder form and at least one polymer selected from the group consisting of natural or synthetic proteins, natural or synthetic oligosaccharides, natural or synthetic polysaccharides, the derivatives thereof and the mixtures thereof;
b) a step of supplying said premix formed in step a) to be fed into a Pharma 11 type extruder of Thermo-Fischer®;
c) a step of mixing, in said extruder, said premixture to obtain a mixture;
d) a step of hot extrusion thermoforming said mixture obtained in step c) in said extruder to obtain a thermoformed extrudate, the hot extrusion step being carried out at a rotation speed of a extrusion screw of 100 rpm and at a temperature comprised between 40° C. and 180° C.;
e) a step of cooling at the outlet of the extruder said thermoformed extrudate obtained in step d); and
f) a step of cutting/grinding, at a grinder, the cooled thermoformed extrudate obtained in step e) in such a way to obtain a homogeneous powder.

The hot extrusion temperature (thermoforming temperature) at which the hot extrusion step is carried out is determined by the type of constituents used, in particular according to the type of polymer and/or plasticizer used, which the person skilled in the art is able to determine. Moreover, a person skilled in the art, in particular according to the type of extruder employed and in accordance with the general principle of the hot extrusion (HME), is able to define possible temperature steps in different zones along the extrusion screw(s) such that a gradual increase in the temperature within the material transported by the extrusion screw(s), this in an advancing direction of the material within the extruder. Typically, between zones defined along the extrusion screws(s), temperature differences in the range of 0 to 40° C. are observed. For example, in the context of the present invention, the below-tested compositions were obtained in a Pharma 11 type extruder of Thermo-Fischer® having 9 temperature zones which are as follows in an advancing direction of the material moving at a speed of 100 rpm: zone 1 (feed zone of the extruder)=ambient temperature; zone 2=120° C.; zone 3=120° C.; zone 4=120° C.; zone 5=130° C.; zone 6=140° C.; zone 7=150° C.; zone 8=155° C.; zone 9 (die)=160° C.

### Example 2: Solubility Test of Thermoformed Compositions According to the Invention Comprising an Extract of Boswellia serrata Standardized to 65% Boswellic Acids

Different thermoformed compositions, obtained according to the manufacturing method described in Example 1, were tested in terms of solubility of boswellic acids present in an extract of Boswellia serrata standardized to 65% boswellic acids. This solubility was measured over time starting from the thermoformed extrudates obtained according to the invention. As indicated above, the thermoformed extrudates are in the form of a homogeneous powder (ground material) in which the triterpene and/or the triterpenoid comprises at least one amorphous phase.

All the solubility tests were carried out with a vane dissolution apparatus, starting with approximately 2 g of thermoformed extrudate, at a temperature of 37° C. under stirring at 50 rpm in 450 ml of a 0.1N HCl dissolution medium. These solubility tests were carried out according to the recommendations of the pharmacopoeia Ph.Eur.9.0 (Recommendations on Dissolution Testing). At determined times (after 30 min and after 2 h), a sample of 1 ml of mixture was taken to carry out a solubility test.

In order to carry out the solubility tests, the tested sample was filtered through a filter (PET, pore size of 0.45 μm, Macherey Nagel) before HPLC analysis (Luna column 5 μm C18(2) 100 A. 100*3 mm (Phenomenex); mobile phase: 85% A: methanol and 15% B: water/acetonitrile (95:5) at pH 2.8; flow rate: 0.6 mL/min, loop: 10 μl, t°=40° C.; wavelengths: 210 nm and 247 nm).

In practice, the solubility of triterpenes and/or of triterpenoids, in particular the solubility of boswellic acids contained in an extract of Boswellia serrata standardized to 65% boswellic acids, was evaluated by HPLC assay of the 6 main boswellic acids (α-boswellic acid, β-boswellic acid, 11-keto-β-boswellic acid (KBA), acetyl-11-keto-β-boswellic acid (AKBA), acetyl α-boswellic acid, acetyl β-boswellic acid) present in this extract of Boswellia serrata.

The thermoformed compositions according to the invention listed in Table 1 were formulated according to the method of the invention and tested in terms of solubility over time according to the principle indicated above (assay of the 6 main boswellic acids: the results presented are the solubility averages calculated by summing the solubilities of each of the 6 boswellic acids and dividing this sum by 6). A single extract of Boswellia serrata standardized to 65% boswellic acids in native crystalline form and in powder form (native BVV) was used as a control. The amounts mentioned in Table 1 are percentages by weight of the compounds used (subjected to the method according to the invention) based on the total weight of the composition.

TABLE 1

| | Extract of | | |
|---|---|---|---|
| | Boswellia serrata | Glycerol | |
| | (65%) (1) | (2) | Protein |
| Compo 1 | 20 | 10 | 70 (3) |
| Compo 2 | 20 | 10 | 70 (4) |
| Compo 3 | 20 | 10 | 70 (5) |

(1) Dry extract of Boswellia serrata standardized to 65% boswellic acids (Vidya Herbs)
(2) Glycerol (Sigma-Aldrich)
(3) Fish collagen with a molecular weight of 3000 Da (Green Snow)
(4) Hydrolyzed fish collagen with a molecular weight below 3000 Da (Kenney & Ross Ltd.)
(5) Bovine gelatin with a molecular weight comprised between 1000 and 3000 Da (Lapi geltine S.P.A)

The results obtained are presented in FIG. 1. As can be seen, the natural proteins of collagen/gelatin type, used as a polymer, allow increasing the solubility of triterpenes and triterpenoids, in particular of boswellic acids. It should be noted that the solubility of boswellic acids varies according to the type/nature of collagen/gelatin but that this solubility is always increased compared to the control.

### Example 3: Dispersion Test of Thermoformed Compositions According to the Invention Comprising an Extract of Boswellia serrata Standardized to 65% Boswellic Acids

Different thermoformed compositions, obtained according to the manufacturing method described in Example 1, were tested in terms of dispersion of the boswellic acids present in an extract of Boswellia serrata standardized to 65% boswellic acids. The dispersion was measured over time starting from the thermoformed extrudates obtained according to the invention. As indicated above, the thermoformed extrudates are in the form of a homogeneous powder (ground material) in which triterpene and/or triterpenoid comprises at least one amorphous phase.

All the dispersion tests were carried out with a vane dissolution apparatus starting with approximately 2 g of thermoformed extrudate, at a temperature of 37° C. under stirring at 50 rpm in 450 ml of a 0.1N HCl dissolution medium.

In order to carry out the dispersion tests, samples taken at determined times (after 30 min and after 2 h) were diluted in an appropriate solvent (mobile phase for HPLC) then filtered through a filter (PET, pore size of 0.45 μm, Macherey Nagel) before HPLC analysis (Luna column 5 μm C18(2) 100 A. 100*3 mm (Phenomenex); mobile phase: 85% A: methanol and 15% B: water/acetonitrile (95:5) at pH 2.8; flow rate: 0.6 mL/min, loop: 10 μl, t°=40° C.; wavelengths: 210 nm and 247 nm).

In practice, the dispersion of triterpenes and/or triterpenoids, in particular the dispersion of boswellic acids contained in an extract of Boswellia serrata standardized to 65% boswellic acids, was evaluated by HPLC assay of the 6 main boswellic acids (α-boswellic acid, β-boswellic acid, 11-keto-β-boswellic acid (KBA), acetyl-11-keto-β-boswellic acid (AKBA), acetyl α-boswellic acid, acetyl β-boswellic acid) present in this extract of Boswellia serrata.

The thermoformed compositions according to the invention listed in Table 2 were formulated according to the method of the invention and tested in terms of aqueous dispersion over time according to the principle indicated above (assay of the 6 main boswellic acids: the results presented are the dispersion averages calculated by summing the dispersions of each of the 6 boswellic acids and dividing this sum by 6). A single extract of Boswellia serrata standardized to 65% boswellic acids in native crystalline form and in powder form (native BVV) was used as a control. The amounts mentioned in Table 2 are percentages by weight of the compounds used (subjected to the method according to the invention) based on the total weight of the composition.

TABLE 2

| | Extract of | | | |
|---|---|---|---|---|
| | Boswellia | | | |
| | serrata | Glycerol | | |
| | (65%) (1) | (2) | Protein | Polysaccharide |
| Compo 1 | 25 | 20 | 0 | 55 (3) |
| Compo 2 | 25 | 20 | 70 (4) | 0 |
| Compo 3 | 25 | 20 | 10 (5) | 45 (3) |
| Compo 4 | 35 | 10 | 55 (6) | 0 |
| Compo 5 | 35 | 10 | 47 (6) | 8 (3) |

(1) Dry extract of Boswellia serrata standardized to 65% boswellic acids (Vidya Herbs)
(2) Glycerol (Sigma-Aldrich)
(3) modified starch Cleargum CB90 (Roquette)
(4) Rice protein (Green Snow)
(5) Pumpkin seed protein (Green Snow)
(6) 5000 Da-hydrolyzed collagen (Rousselot)

The obtained results are presented in FIG. 2 for compositions 1 to 3 and in FIG. 3 for compositions 4 and 5. As can be seen, each of the thermoformed compositions according to the invention gives rise to a percentage of dispersion significantly greater than that observed for the single extract of Boswellia serrata standardized to 65% boswellic acids in native crystalline form and in powder form (native BW).

### Example 4: Solubility Test of Thermoformed Compositions According to the Invention Comprising an Extract of Bacopa monierii Standardized to 20% Bacosides

Different thermoformed compositions, obtained according to the manufacturing method described in Example 1, were tested in terms of solubility of bacosides present in an extract of Bacopa monierii standardized to 20% bacosides. This solubility was measured over time starting from the thermoformed extrudates obtained according to the invention. As indicated above, the thermoformed extrudates are in the form of a homogeneous powder (ground material) in which triterpene and/or triterpenoid comprises at least one amorphous phase.

All the solubility tests were carried out with a vane dissolution apparatus starting with approximately 4 g of thermoformed extrudate, at a temperature of 37° C. under stirring at 50 rpm in 900 ml of a 0.1N HCl dissolution medium. These solubility tests were carried out according to the recommendations of the pharmacopoeia Ph.Eur.9.0 (Recommendations on Dissolution Testing). At determined times (after 30 min and after 2 h), a sample of 1 ml of mixture was taken to carry out a solubility test.

In order to carry out the solubility tests, the tested sample was centrifuged (10000 rpm for 10 min at room temperature, Microstar 17 (VWR)), the supernatant was filtered through a filter (PET, pore size of 0.45 μm, Macherey Nagel) before HPLC analysis (C18 column, 5 μm 250*4.6 mm (Agilent); mobile phase: A: 0.1 mM phosphate buffer and B: Acetonitrile according to the following gradient:

| TIME (MIN) | A | B |
|---|---|---|
| 0 | 70% | 30% |
| 25 | 60% | 40% |
| 26 | 70% | 30% |
| 30 | 70% | 30% |

Flow rate: 1.5 ml/min; loop, 20 μl; t°: 25° C.; wavelength 205 nm.

In practice, the solubility of triterpenes and/or of triterpenoids, in particular the solubility of bacosides contained in an extract of Bacopa monierii standardized to 20% bacosides, was evaluated by HPLC assay of the 5 main bacosides (Bacopaside I, Bacoside A3, Bacopaside II, Jujubogenin, Bacopasaponin C) present in this extract of Bacopa monierii.

The thermoformed compositions according to the invention listed in Table 3 were formulated according to the method of the invention and tested in terms of solubility over time according to the principle indicated above (assay of the 5 main bacosides: the results presented are the solubility averages calculated by summing the solubilities of each of the 5 bacosides and dividing this sum by 5). A single extract of Bacopa monierii standardized to 20% bacosides in native crystalline form and in powder form (native BC) was used as a control. The amounts mentioned in Table 3 are percentages by weight of the compounds used (subjected to the method according to the invention) based on the total weight of the composition.

TABLE 3

| | Extract of | | | |
|---|---|---|---|---|
| | Bacopa | | | |
| | Monierii | Glycerol | Protein | Polysaccharide |
| | (20%) (1) | (2) | (3) | (4) |
| Compo 1 | 33 | 14 | 53 | 0 |
| Compo 2 | 33 | 14 | 43 | 10 |
| Compo 3 | 25 | 14 | 0 | 53 |

(1) Dry extract of Bacopa Monierii standardized to 20% bacosides (Vidya Herbs)
(2) Glycerol (Sigma-Aldrich)
(3) Hydrolyzed collagen with a molecular weight of 5000 Da (Rousselot)
(4) Tackidex C760 (Roquette)

The results obtained are presented in FIG. 4. As can be seen, all the compositions according to the invention allow increasing the % of solubilized bacosides relative to the control.

### Example 5: Solubility Test of Thermoformed Compositions According to the Invention Comprising Asiaticoside

Different thermoformed compositions, obtained according to the manufacturing method described in Example 1, were tested in terms of solubility of the asiaticoside. This solubility was measured over time starting from the thermoformed extrudates obtained according to the invention. As indicated above, the thermoformed extrudates are in the form of a homogeneous powder (ground material) in which triterpene and/or triterpenoid comprises at least one amorphous phase.

All the solubility tests were carried out with a vane dissolution apparatus starting with approximately 2 g of thermoformed extrudate, at a temperature of 37° C. under stirring at 50 rpm in 900 ml of a 0.1N HCl dissolution medium. These solubility tests were carried out according to the recommendations of the pharmacopoeia Ph.Eur.9.0 (Recommendations on Dissolution Testing). At determined times (after 30 min and after 2 h), a sample of 1 ml of mixture was taken to carry out a solubility test.

In order to carry out the solubility tests, the tested sample was centrifuged (10000 rpm for 10 min at room temperature, Microstar 17(VWR)), the supernatant was filtered through a filter (PET, pore size of 0.45 μm, Macherey Nagel) before HPLC analysis (C18 column, 5 μm 250*4.6 mm (Agilent); mobile phase: A: Water and B: Acetonitrile according to the following gradient:

| TIME (MIN) | A | B |
|---|---|---|
| 0 | 80% | 20% |
| 18 | 59% | 41% |
| 20 | 45% | 55% |
| 23 | 45% | 55% |
| 28 | 80% | 20% |
| 33 | 80% | 20% |

Flow rate: 1 ml/min; loop, 10 μl; t°: 25° C.; wavelength 205 nm).

In practice, the solubility of triterpenes and/or triterpenoids, in particular the solubility of asiaticoside, was evaluated by HPLC assay.

The thermoformed compositions according to the invention listed in Table 4 were formulated according to the method of the invention and tested in terms of solubility over time according to the principle indicated above. Asiaticoside in native crystalline form and in powder form (native AS) was used as a control. The amounts mentioned in Table 4 are percentages by weight of the compounds used (subjected to the method according to the invention) based on the total weight of the composition.

TABLE 4

| | Asiaticoside | Glycerol | Protein | Polysaccharide |
|---|---|---|---|---|
| | (1) | (2) | (3) | (4) |
| Compo 1 | 10 | 10 | 70 | 10 |
| Compo 2 | 10 | 14 | 0 | 76 |

(1) Asiaticoside (FyzCo)
(2) Glycerol (Sigma-Aldrich)
(3) Hydrolyzed collagen with a molecular weight of 5000 Da (Rousselot)
(4) Tackidex C760 (Roquette)

The results obtained are presented in FIG. 5. As can be seen, all the compositions according to the invention allow increasing the % of solubilized asiaticoside relative to the control.

### Example 6: Dispersion Test of Thermoformed Compositions According to the Invention Comprising Ursolic Acid

Different thermoformed compositions, obtained according to the manufacturing method described in Example 1, were tested in terms of dispersion of ursolic acid. The dispersion was measured over time starting from the thermoformed extrudates obtained according to the invention. As indicated above, the thermoformed extrudates are in the form of a homogeneous powder (ground material) in which triterpene and/or triterpenoid comprises at least one amorphous phase.

All the dispersion tests were carried out with a vane dissolution apparatus starting with approximately 2 g of thermoformed extrudate, at a temperature of 37° C. under stirring at 50 rpm in 900 ml of an 0.1N HCl dissolution medium.

In order to carry out the dispersion tests, samples taken at determined times (after 30 min and after 2 h) were diluted in an appropriate solvent (mobile phase for HPLC) then filtered through a filter (PET, pore size of 0.45 μm, Macherey Nagel) before HPLC analysis (Luna column 5 μm C18(2) 100 A. 100*3 mm (Phenomenex); mobile phase Acetonitrile/Water/0.5% Ammonium acetate (67:12:21) flow rate: 1 mL/min, loop: 10 μl, t°=25° C.; wavelengths: 210 nm).

In practice, the dispersion of triterpenes and/or triterpenoids, in particular the dispersion of ursolic acid, was evaluated by HPLC assay.

The thermoformed compositions according to the invention listed in Table 5 were formulated according to the method of the invention and tested in terms of aqueous dispersion over time according to the principle indicated above. Ursolic acid in native crystalline form and in powder form (native AU) was used as a control. The amounts mentioned in Table 5 are percentages by weight of the compounds used (subjected to the method according to the invention) based on the total weight of the composition.

TABLE 5

| | Ursolic acid | Glycerol | Protein | Polysaccharide |
|---|---|---|---|---|
| | (1) | (2) | (3) | (4) |
| Compo 1 | 10 | 10 | 80 | 0 |
| Compo 2 | 10 | 14 | 66 | 10 |
| Compo 3 | 10 | 14 | 0 | 76 |

(1) Ursolic acid (Fyzco)
(2) Glycerol (Sigma-Aldrich)
(3) Hydrolyzed collagen with a molecular weight of 5000 Da (Rousselot)
(4) Tackidex C760 (Roquette)

The results obtained are presented in FIG. 6. As can be seen, all the compositions according to the invention allow increasing the % of dispersed ursolic acid relative to the control.

The present invention has been described in relation to the specific embodiments, which have a purely illustrative value and should not be considered as limitative. In general, it will appear obvious to the person skilled in the art that the present invention is not limited to the examples illustrated and/or described above.

The use of the verbs “comprise”, “include”, or any other variant, as well as the conjugations thereof, can in no way exclude the presence of elements other than those mentioned.

The use of the indefinite article “a”, “an”, or of the definite article “the”, to introduce an element does not exclude the presence of a plurality of these elements.

1. A composition in the form of a thermoformed extrudate comprising at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.

2. The composition according to claim 1, characterized in that said thermoformed extrudate comprises a thermoformed mixture of said at least one triterpene and/or of said at least one triterpenoid and/or of said at least one of the glycosylated forms thereof and of said at least one polymer selected from the group consisting of natural or synthetic proteins, natural or synthetic oligosaccharides, natural or synthetic polysaccharides, the derivatives thereof and the mixtures thereof.

3. The composition according to claim 1, characterized in that said at least one triterpene and/or said at least one triterpenoid and/or said at least one of the glycosylated forms thereof predominantly comprises at least one first amorphous phase.

4. The composition according to claim 3, characterized in that said at least one triterpene and/or said at least one triterpenoid and/or said at least one of the glycosylated forms thereof comprises between 51 and 100% by mass of an amorphous phase and between 0 and 49% by mass of a crystalline phase.

5. The composition according to claim 1, characterized in that said at least one triterpene and/or said at least one triterpenoid and/or said at least one of the glycosylated forms thereof is tetracyclic, such as for example oleandrine, euphol or cucurbitacin, or pentacyclic, such as for example betulinic acid, oleanolic acid, boswellic acid, ursolic acid, lupeol, asiatic acid, madecassic acid, maslinic acid, jujubogenin or pseudojujubogenin.

6. The composition according to claim 5, characterized in that said boswellic acid is selected from the group consisting of α- and β-boswellic acids, for example α-boswellic acid, acetyl-α-boswellic acid, β-boswellic acid, acetyl-β-boswellic acid, 9,11-dehydro-α-boswellic acid, acetyl-9,11-dehydro-α-boswellic acid, 9,11-dehydro-β-boswellic acid, acetyl-9,11-dehydro-β-boswellic acid, 11-keto-β-boswellic acid, 11-keto-α-boswellic acid, 3-acetyl-11-keto-α-boswellic acid and 3-acetyl-11-keto-β-boswellic acid.

7. The composition according to claim 1, characterized in that said natural or synthetic proteins are selected from the group consisting of glycoproteins, collagens and/or collagen hydrolysates, plant proteins, animal proteins, the derivatives thereof and the mixtures thereof.

8. The composition according to claim 7, characterized in that said collagens and/or said collagen hydrolysates have a molecular weight comprised between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

9. The composition according to claim 1, characterized in that said oligosaccharides are selected from the group consisting of cyclodextrin, raffinose, rhamminose, rhamnose, stachyose, verbascose, trehalose, lactose, lactulose, maltose, the derivatives thereof and the mixtures thereof.

10. The composition according to claim 1, characterized in that said natural or synthetic polysaccharides are selected from the group consisting of starches, fibers, celluloses, hemicelluloses, glycogen, β-glucan, inulin, amylopectin, amylose, dextrin, maltodextrin, isomaltose, xylan, pullulan, agar-agar, carrageenans, mannans, fucoidan, gums, chitosan, chitin, xanthan, levan, neoserine, hyaluronic acid, hyaluronates, chondroitin sulphate, dermatan sulphate, keratan sulphate, the derivatives thereof and the mixtures thereof.

11. The composition according to claim 1, further comprising at least one additional natural or synthetic polymer selected from the group consisting of polyvinyl acetate, polyvinylpyrrolidone (PVP), polyvinylpyrrolidone-co-vinyl acetate, polyethylene-co-vinyl acetate, polyvinyl acid co-methacrylic acetate, polyethylene oxide, polylactide-co-glycolide, polyvinyl alcohol, polycarbophil, polycaprolactone, carnauba wax, ethylene-vinyl copolymer, lecithin, castor oil, hydrogenated soybean oil, waxes, isomalt, the derivatives thereof and the mixtures thereof.

12. The composition according to claim 1, further comprising at least one plasticizer.

13. The composition according to claim 12, characterized in that said at least one plasticizer is selected from the group consisting of polyols, lipids, sucrose esters, water, triethyl citrate, polyethylene glycol, dibutyl sebate, butyl stearate, glycerol monostearate, diethyl phthalate, the derivatives thereof and the mixtures thereof.

14. The composition according to claim 1, further comprising at least one additive selected from the group consisting of lubricants, surfactants, antioxidants, chelants, the derivatives thereof and the mixtures thereof.

15. The composition according to claim 1, further comprising at least one first additional compound of polyphenol type selected from the group consisting of phenolic acids, stilbenes, phenolic alcohols, lignans, flavonoids, the derivatives thereof and the mixtures thereof.

16. The composition according to claim 1, characterized in that it is packaged in the form of pellets, flakes, granules, powders, effervescent or non-effervescent tablets, injectable or non-injectable solutions, suspensions, gels, ointments or even in any other suitable form allowing administration to an animal or a human being.

17. A manufacturing method, in particular a method for manufacturing by thermoforming, a composition in the form of a thermoformed extrudate according to claim 1, characterized in that it comprises the following steps:
a) a step of simultaneous or delayed supplying at least one triterpene and/or at least one triterpenoid and/or at least one of the glycosylated forms thereof and at least one polymer selected from the group consisting of natural or synthetic proteins, natural or synthetic oligosaccharides, natural or synthetic polysaccharides, the derivatives thereof and the mixtures thereof, to be fed into an extruder,
b) a step of mixing, in said extruder, said at least one triterpene and/or said at least one triterpenoid and/or said at least one of the glycosylated forms thereof and said at least one polymer selected from the group consisting of natural or synthetic proteins, natural or synthetic oligosaccharides, natural or synthetic polysaccharides, the derivatives thereof and the mixtures thereof, to form a mixture, and
c) a step of hot extruding said mixture obtained in step b) in said extruder to obtain a thermoformed extrudate in which said at least one triterpene and/or said at least one triterpenoid and/or said at least one of the glycosylated forms thereof comprises/comprise at least one first amorphous phase and optionally one second crystalline phase.

18. The method according to claim 17, characterized in that it comprises a preliminary step of premixing said at least one triterpene and/or said at least one triterpenoid and/or said at least one of the glycosylated forms thereof and said at least one polymer selected from the group consisting of natural or synthetic proteins, natural or synthetic oligosaccharides, natural or synthetic polysaccharides, the derivatives thereof and the mixtures thereof, in such a way to form a premixture intended to be fed into the extruder.

19. The method according to claim 17, characterized in that said hot extrusion step is carried out at an extrusion temperature comprised between 20 and 300° C., preferably at a temperature comprised between 40 and 270° C., preferentially at a temperature comprised between 50 and 250° C., preferably at a temperature comprised between 60 and 230° C., more preferentially at a temperature comprised between 70 and 220° C., more preferentially at a temperature comprised between 80 and 200° C., more preferentially at a temperature comprised between 90 and 180° C., more preferentially at a temperature comprised between 100 and 170° C., more preferentially at a temperature comprised between 120 and 160° C., more preferentially at a temperature comprised between 125 and 150° C.

20. The method according to claim 17, characterized in that said hot extrusion step is carried out at a rotation speed of an extrusion screw comprised between 20 and 900 rpm, preferably comprised between 50 and 300 rpm, preferably comprised between 100 and 250 rpm, preferentially equal to 250 rpm, preferentially equal to 100 rpm.

21. The method according to claim 17, characterized in that it comprises an additional step of cooling at the outlet of the extruder.

22. The method according to claim 17, characterized in that it comprises an additional step of treating the thermoformed extrudate at the outlet of the extruder, for example cutting at a pelletizer and/or grinding said thermoformed extrudate.

23. The composition in the form of a thermoformed extrudate according to claim 1 for use in the preventive and/or curative treatment, in human and/or in animal, of pathologies related to inflammations, pathologies related to the premature aging of cells, pathologies related to the cardiovascular system, pathologies related to the blood system, pathologies related to the gastrointestinal system, pathologies related to the endocrine system, pathologies related to the immune system, pathologies related to the central nervous system, skin diseases, diseases due to the presence of microorganisms and cancers and in the preventive and/or curative treatment of diabetes.

24. The composition in the form of a thermoformed extrudate obtained according to the method according to claim 17, said composition comprising at least one triterpene and/or at least one triterpenoid and/or at least one of the glycosylated forms thereof such as active ingredient and at least one polymer selected from the group consisting of natural or synthetic proteins, natural or synthetic oligosaccharides, natural or synthetic polysaccharides, the derivatives thereof and the mixtures thereof, said at least one triterpene and/or said at least one triterpenoid and/or said at least one of the glycosylated forms thereof comprising at least one first amorphous phase and optionally one second crystalline phase.
